(19) United States
(12) Patent Application Publication
(10) Pub. No.: US 2007/0251530 A1
(43) Pub. Date: Nov. 1, 2007

(54) SCREENING CANDIDATES FOR IMPLANTABLE INFUSION DEVICES

(75) Inventor: Keith R. Hildebrand, Houlton, WI (US)

Correspondence Address: MEDTRONIC, INC., 710 MEDTRONIC PARKWAY NE, MINNEAPOLIS, MN, 55432-9924, US

(73) Assignee: Medtronic, Inc., Minneapolis, MN (US)

(21) Appl. No.: 11/380,698

(22) Filed: Apr. 28, 2006

(51) Int. Cl. A61M 5/14

(52) U.S. Cl. 128/898; 604/891.1

(57) ABSTRACT

Pharmaceutical depots and methods for screening patients to identify good candidates for implantable infusion device therapy are described. A pharmaceutical depot is placed in a target region of a patient of a pharmaceutical depot configured to release a therapeutic agent in a manner similar to what would be achieved by an implantable infusion system, e.g. small amounts over a period of time. Following depot placement, the patient can be evaluated to determine whether at least one symptom of a disease or general quality of life has improved. If an improvement results, the patient is identified as a good candidate for receiving an implantable infusion device to deliver the therapeutic agent to the target region to treat the disease.

### FIELD

This invention relates to medical devices, compositions, and methods for treating and selecting patients, and more particularly for identifying patients for which implantable infusion device therapy may be beneficial.

### BACKGROUND

Screening for patients that may respond favorably to implantable infusion device therapies is a challenging and variable procedure and it would be desirable improve the ability to predict long-term success of such implantable therapies.

Implantable infusion devices contain a reservoir from which therapeutic agent may be delivered over a period of time. In many situations, the implantable infusion device is implanted subcutaneously in a patient's abdominal cavity. A proximal end of a catheter may be operably coupled to the infusion device, and an infusion portion (typically the distal portion) of the catheter is placed in proximity to a region of the patient to which therapeutic agent is desired to be delivered. Often, as with baclofen for treatment of spasticity and morphine for treatment of pain, the therapeutic agent is delivered to the intrathecal space of the patient. Such infusion systems are typically chronically implanted in the patient and are designed to chronically deliver therapeutic agent to the patient. Such systems can provide high levels of efficacy in otherwise treatment-resistant patients and can dramatically increase the quality of life of patients having such systems implanted.

However, not all potential patients will receive the high degree of efficacy associated with implantable infusion therapy. Furthermore, the expense of the devices and surgery associated with implanting infusion systems, as well as the risks associated with such surgery, warrant a sufficient screening procedure to ensure that implantation of an infusion device is likely to be effective.

In some instances, a single injection is used as a basis of screening. Of course, a single injection in many cases may not serve as an accurate predictor of how a patient will respond to chronic delivery of a therapeutic agent via an implanted infusion system.

In other instances, external pumps are used in trialing systems to determine whether a patient may respond favorably to implantable infusion device therapy. In trialing for agents to be administered intrathecally via an implantable infusion device, a catheter is placed percutaneously such that an infusion section of the catheter is located epidurally or intrathecally and the proximal end of the catheter is coupled to an external pump. The external pump delivers the agent intraspinally through the catheter for a period of time, typically one day to one month. With such trialing systems, the longer the trialing period, the greater the risk of complications, e.g. from infection. The shorter the trialing period, the less likely that the trial will be effective to determine whether a patient may benefit from an implanted system that will chronically deliver the therapeutic agent. In addition, the presence of an external pump for an extended period of time can be awkward for the patient.

It would be desirable to have an alternative, yet accurate, reliable and minimally invasive screening procedure to determine whether a patient would be a good candidate for implantable infusion device therapy.

### BRIEF SUMMARY

This disclosure describes methods and pharmaceutical depots useful for screening whether a patient would be a good candidate to receive an implantable infusion device as part of a therapy to treat a disease, disorder or condition of the patient. The depots are configured to release therapeutic agents over a period of time, e.g. up to a month or two, and to closely mimic implantable infusion device delivery profile, i.e. small amounts of therapeutic agent over a sustained period.

In one aspect, this disclosure discusses a method for identifying whether a patient having a disease is a candidate for delivery of a therapeutic agent via an implantable infusion device to a targeted location for treatment of the disease. The method comprises placing a pharmaceutical depot comprising the therapeutic agent in the patient. Preferably, the depot is configured to release the therapeutic agent in a manner substantially similar to what would be delivered by an implantable infusion device, e.g. a constant rate for about a week. The depot is placed in proximity to the targeted location. Following implantation of the depot, the patient is monitored to determine whether the symptom has improved. If the symptom has improved, the patient is identified as a candidate for delivery of a therapeutic agent via an implantable infusion device.

Various embodiments described in this disclosure may provide one or more advantages over existing screening devices, compositions and methods. For example, use of a pharmaceutical depot as a screening agent more closely mimics implantable infusion device delivery compared to a single injection, e.g. small amounts of drug over a sustained period. In addition, use of a pharmaceutical depot as a screening agent avoids complications and expense associated with ambulatory pump trials for screening whether a patient is a good candidate to receive therapy via an implantable infusion system. These and other advantages will be apparent to one of skill in the art upon reading the disclosure.

### BRIEF DESCRIPTION OF THE DRAWINGS

FIGS. 1-6 are flow charts of screening methods.

FIG. 7 is a diagrammatic illustration of an infusion system.

FIG. 8 is a diagrammatic illustration of an infusion device and a catheter implanted in a patient.

The figures are not necessarily to scale.

### DETAILED DESCRIPTION

In the following descriptions, reference is made to the accompanying drawings that form a part hereof, and in which are shown by way of illustration several specific embodiments of the invention. It is to be understood that other embodiments of the present invention are contemplated and may be made without departing from the scope or spirit of the present invention. The following detailed description, therefore, is not to be taken in a limiting sense.

This disclosure provides a discussion of various methods of screening whether a patient would be a good candidate to receive an implantable infusion device as part of a therapy to treat a disease of the patient. The methods generally include placing in a patient a pharmaceutical depot comprising the therapeutic agent. Preferably, the depot is placed in proximity to a target location to which the pharmaceutical agent would be delivered if the patient were to receive an implantable infusion device. The depot would be configured to release therapeutic agent over a period of time to approximate delivery profile from an implantable infusion device, e.g. small amounts of therapeutic agent over a sustained period. After implantation of the depot, a physician, other health care provider or the patient may determine whether a symptom of the disease, or general quality of life of the patient, has improved. If the symptom or quality of life has improved, the patient is considered a good candidate for receiving an implantable infusion system to deliver the agent to the target location. The patient and physician will be better able to determine whether receiving the implantable infusion system is warranted.

Definitions

All scientific and technical terms used in this application have meanings commonly used in the art unless otherwise specified. The definitions provided herein are to facilitate understanding of certain terms used frequently herein and are not meant to limit the scope of the present disclosure.

In the context of the present disclosure, the terms “treat”, “therapy”, and the like mean alleviating, slowing the progression, preventing, attenuating, or curing the treated disease or a symptom of the treated disease.

As used herein, “disease”, “disorder”, “condition” and the like, as they relate to a subject's health, are used interchangeably and have meanings ascribed to each and all of such terms.

As used herein, a “symptom” of a disease means subjective or objective evidence of the disease. For example, a symptom of pain may be a subjective determination of the patient as to how much pain the patient is feeling, a symptom of essential tremor may be the degree of tremor in a patient's extended hand, a symptom of Alzheimer's may be the degree of plaque formation in the patient's brain, and the like.

As used herein, “bioerodable”, “biodegradable” and the like, as they relate to pharmaceutical depots, are used interchangeable and have meanings ascribed to each and all of such terms.

It is noted that the terms “comprises” and variations thereof do not have a limiting meaning where these terms appear in the accompanying description and claims. Moreover, “a,” “an,” “the,” “at least one,” and “one or more” are used interchangeably herein.

1. Screening

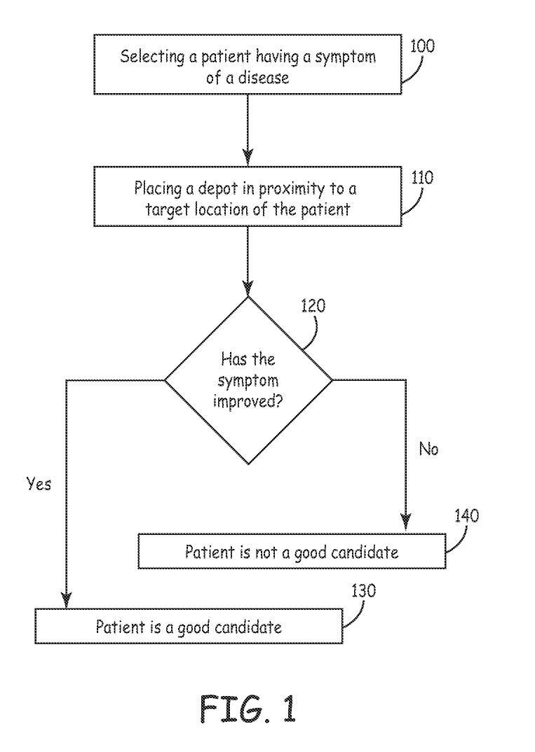

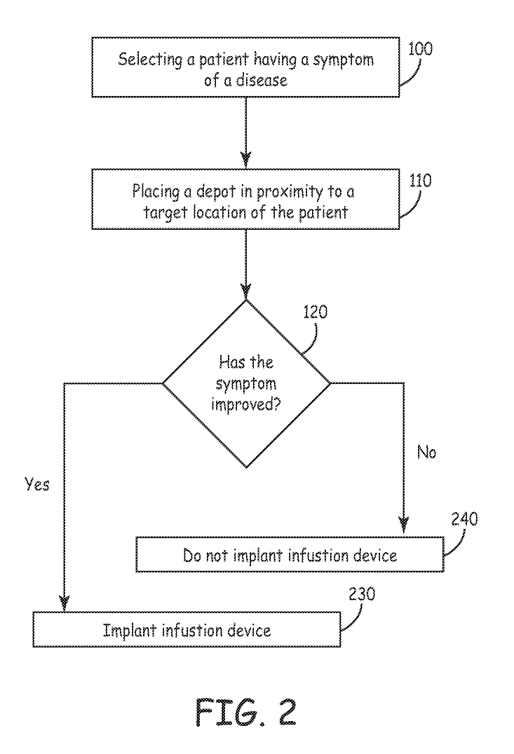

FIGS. 1 and 2 illustrate screening methods that may be employed to determine whether a patient having a disease is a candidate for delivery of a therapeutic agent via an implantable infusion device to a targeted location for treatment of the disease. As shown in FIG. 1, a patient having a symptom of a disease is selected (100). A depot comprising a therapeutic agent that may be useful for treating the disease is placed in proximity to a target location of the patient (110). The patient is then evaluated to determine whether the symptom has improved (120). If the symptom has improved the patient is considered to be a good candidate to receive an implantable infusion device to deliver the therapeutic agent to the target location of the patient to treat the disease (130). As shown in FIG. 2, an implantable infusion device may be implanted in the patient (230) if the symptom has improved (120).

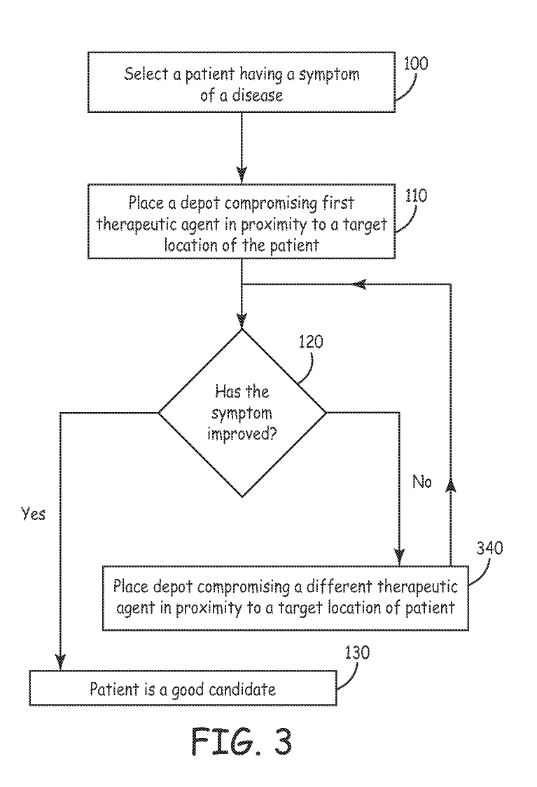

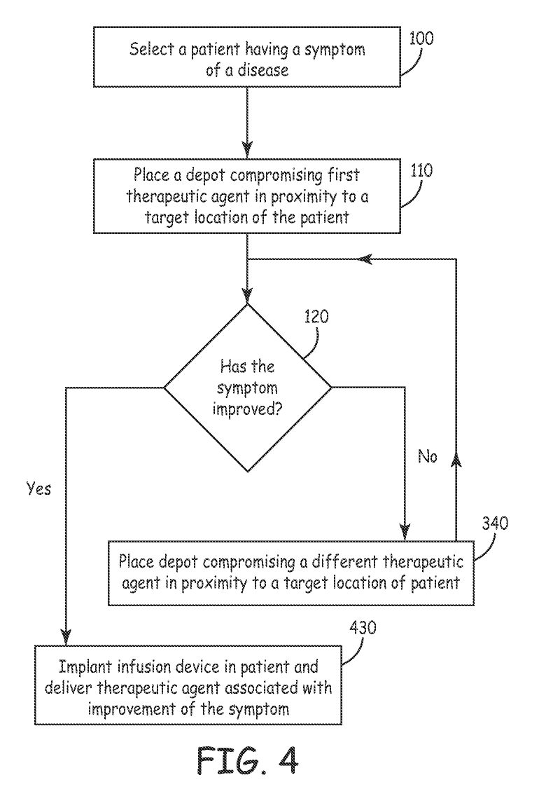

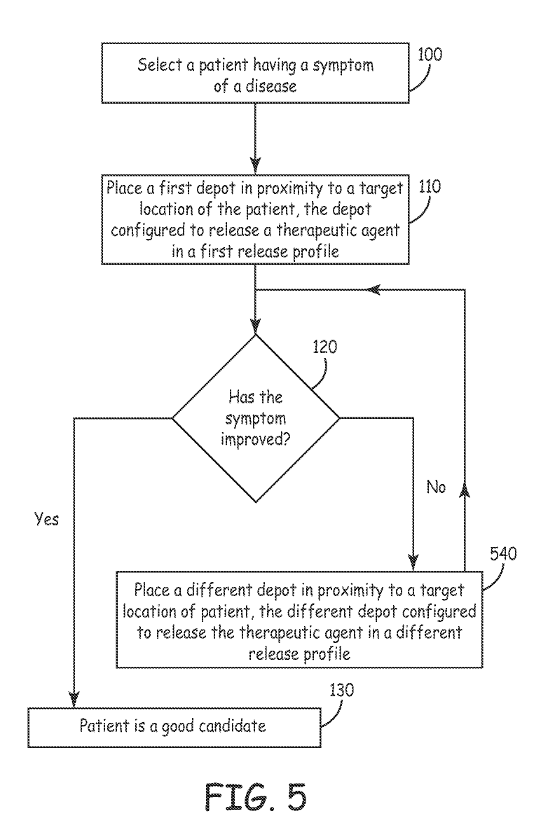

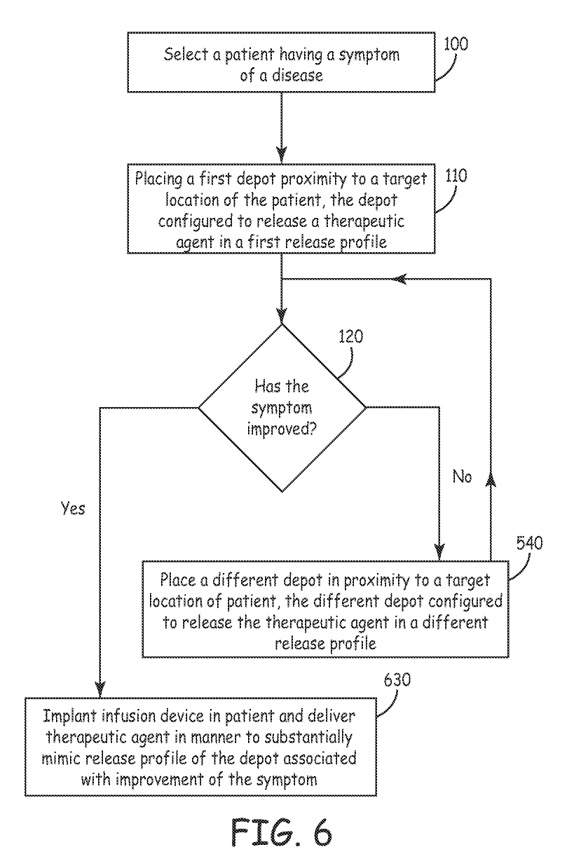

FIGS. 3-6 illustrate methods where different depots may be employed as screening agents should the patient not respond to a first or prior depot. As shown in FIGS. 3 and 4, if the symptom has not improved (120), a depot comprising a different therapeutic agent may be placed in proximity to the target location of patient. If the symptom improves (120), the patient may be implanted with an infusion system that delivers the different therapeutic agent (430). As shown in FIGS. 5 and 6, if the symptom has not improved (120), a different depot configured to release the therapeutic agent in a different release profile may be placed in proximity to a target location of patient (540). If the symptom improves (120), the patient may be implanted with an infusion system configured to deliver the therapeutic agent in manner to substantially mimic the release profile of the different depot (530).

2. Pharmaceutical Depot

One or more therapeutic agents may be placed in a pharmaceutical depot and used as a screening agent. Of course, it will be understood that the depots may themselves be used as a means for providing therapy to treat a disease. Suitable depots may take the form of capsules, microspheres, particles, rods, gels, coatings, matrices, wafers, pills, and the like.

A depot may comprise a biopolymer. The biopolymer may be a sustained-release biopolymer and may be biodegradable. The depot may be deposited at or near, generally in close proximity, to a target site, such as a spinal or perispinal location. Examples of suitable sustained release biopolymers include but are not limited to poly(alpha-hydroxy acids), poly(lactide-co-glycolide) (PLGA), polylactide (PLA), polyglycolide (PG), polyethylene glycol (PEG) conjugates of poly(alpha-hydroxy acids), polyorthoesters, polyaspirins, polyphosphagenes, collagen, starch, chitosans, gelatin, alginates, dextrans, vinylpyrrolidone, polyvinyl alcohol (PVA), PVA-g-PLGA, PEGT-PBT copolymer (polyactive), methacrylates, poly(N-isopropylacrylamide), PEO-PPO-PEO (pluronics), PEO-PPO-PAA copolymers, PLGA-PEO-PLGA, or combinations thereof.

Depots may be deposited in proximity to any location in which a therapeutic agent may be delivered via an implantable pump to treat a disease. For example, if a therapeutic agent may be delivered intrathecally via an implantable pump to treat a disease, the depot may be placed in the epidural, intrathecal, subarachnoid, or subdural spaces. If the depot is placed intrathecally, it may be desirable for the depot to be in the form of a gel, suspension, or the like. If the depot is placed perispinally, e.g. epidurally, but the implantable infusion system would deliver the therapeutic agent intrathecally, the depot may include a penetration enhancer to promote passage from the perispinal space to the intrathecal space. Examples of penetrating enhancers include propylene glycol, alkyl methyl sulfoxides, pyrrolidones, fatty acids such as oleic, lauric and stearic acid, surfactants such as sodium lauryl sulfate, lecithin and isopropyl myristate. Regardless of where the depot is placed, it may be introduced via a needle or cannula and injected or infused into a target region. The screening dosage may be increased by increasing the volume of a particular biodegradable depot formulation that is administered generally without affecting the duration of the screen. In addition, it may be possible to screen for the effects of two or more agents which may be combined in the reservoir of the implantable infusion system by delivering separate injections of specific single-agent depots or combining more than one agent in a given depot formulation.

Depots may be configured to release a therapeutic agent according to a variety of release profiles using any currently available or future developed polymer or combinations of polymers according to any currently available or future developed techniques. Preferably, the depot is configured to release the therapeutic agent according to a profile similar to that which may be achieved when employing an implantable infusion system to deliver the therapeutic agent. For example the depot may be configured to release a substantially constant amount of the therapeutic agent for a period of at least about three days, about five days, about one week, about two weeks, about one month, about two months, or longer, or between about 3 days and about 7 days, after placement of the depot within a patient. In general, the release rate of therapeutic agent from a depot may by altered by selection of the specific biodegradable polymer, the molecular weight of the polymer, and the chemical structure and modifications of the polymer including the number and length of side chains, cross linking, density of hydrolytic sites, and hydrophilicity of the polymer. The release rate of the depot may also be altered by changing the ratio of monomers in copolymer formulations, e.g. the lactide-to-glycolide ratio in PLGA, changing the ratio of the therapeutic agent to the polymer, adding excipients to the polymeric formulation and changing the surface area of the dosage form for surface eroding polymers, and the like. While a constant delivery rate is most likely to mimic infusion from an implantable infusion device, it will be understood that efficacy from the depot may be obtained more quickly with a burst of therapeutic initially. However, it will be further understood that achieving efficacy with a large initial burst of therapeutic agent release may not, in many cases, provide an accurate indication of whether implantable infusion device therapy will be efficacious.

Any therapeutic agent that may be delivered by an implantable infusion device to treat a disease may be placed in a depot and used as a screening tool or as providing therapy in accordance with the teachings of this disclosure. Exemplary agents that may be used include agents for treating spasticity, such as baclofen; and agents for treating pain, such as opioid agonists (including morphine and sufentanil), local anesthetics (including bupivacaine), alpha2-agonists (including clonidine), gabapentin, TNFα inhibitors, norepinephrine and serotonin reuptake inhibitors, and tricyclic antidepressants (including desipramine).

Table 1 provides a representative list of agents that may be placed in a depot and used in accordance with the teachings of this disclosure. Exemplary concentrations, in terms of % loading=(weight of drug/total weight of depot), at which the therapeutic agent may be placed in the depot, as well as a release rate that would closely mimic the delivery profile of therapeutic agent delivered from an implantable infusion system, and a duration for which the therapeutic agent may be release from the depot at the release rate to achieve a good indication of whether the patient may be a good candidate for an implantable infusion device. The implantable infusion device therapy that the depot is designed to closely mimic is also provided in Table 1.

TABLE 1
Concentrations of agents and release profiles from exemplary depots

| Therapeutic | Concentration | | | Duration at | Implantable infusion |
|---|---|---|---|---|---|
| agent | (% loading) | Release rate | | release rate | device therapy |
| baclofen | 1 to 20% | 0.1 to 1.0 | mg/day | 3 days to 2 | Intrathecal for |
| | | | | weeks | treatment of spasticity |
| morphine | 10 to 50% | 1 to 15 | mg/day | 3 days to 2 | Intrathecal for |
| | | | | weeks | treatment of pain |
| sufentanil | 05 to 10% | 1 to 20 | mcg/day | 3 days to 2 | Intrathecal for |
| | | | | weeks | treatment of pain |
| bupivacaine | 5 to 75% | 2 to 25 | mg/day | 3 days to 2 | Intrathecal for |
| | | | | weeks | treatment of pain |
| clonidine | 1 to 20% | 0.1 to 1.0 | mg/day | 3 days to 2 | Intrathecal for |
| | | | | weeks | treatment of pain |
| gabapentin | 5 to 75% | 1 to 50 | mg/day | 3 days to 2 | Intrathecal for |
| | | | | weeks | treatment of pain |
| desipramine | 5 to 75% | 1 to 50 | mg/day | 3 days to 2 | Intrathecal for |
| | | | | weeks | treatment of pain |

Infusion System

Any implantable infusion system may be used in accordance with the teachings of this disclosure. Implantable infusion systems typically comprise an infusion device and a catheter operably coupled to the infusion device. The therapy delivery device may be a pump device. Non-limiting examples of pump devices include osmotic pumps, fixed-rate pumps, programmable pumps and the like. Each of the aforementioned pump systems comprise a reservoir for housing a fluid composition comprising a therapeutic agent. The catheter comprises one or more delivery regions, through which the fluid may be delivered to one or more target regions of the subject.

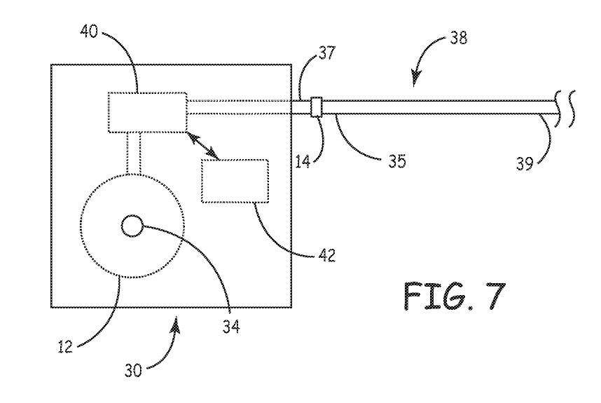

The infusion device 30 shown in FIG. 7 comprises a reservoir 12 for housing a composition comprising a therapeutic agent and a pump 40 operably coupled to the reservoir 12. The catheter 38 shown in FIG. 7 has a proximal end 35 coupled to the therapy delivery device 30 and a distal end 39 adapted to be implanted in a patient.

Between the proximal end 35 and distal end 39 or at the distal end 39, the catheter 38 comprises one or more delivery regions (not shown) through which the therapeutic agent may be delivered. The infusion device 30 may have a port 34 into which a hypodermic needle can be inserted to inject a quantity of therapeutic agent into reservoir 12. The infusion device 30 may have a catheter port 37, to which the proximal end 35 of catheter 38 may be coupled. The catheter port 37 may be operably coupled to reservoir 12. A connector 14 may be used to couple the catheter 38 to the catheter port 37 of the therapy delivery device 30. The infusion device 30 may be operated to discharge a predetermined dosage of the pumped fluid into a target region of a patient. The infusion device 30 may contain a microprocessor 42 or similar device that can be programmed to control the amount of fluid delivery. The programming may be accomplished with an external programmer/control unit via telemetry. A controlled amount of fluid comprising a therapeutic agent may be delivered over a specified time period. With the use of a programmable delivery device 30, different dosage regimens may be programmed for a particular patient. Additionally, different therapeutic dosages can be programmed for different combinations of fluid comprising therapeutics. Those skilled in the art will recognize that a programmed infusion device 30 allows for starting conservatively with lower doses and adjusting to a more aggressive dosing scheme, if warranted, based on safety and efficacy factors.

If it is desirable to administer more than one therapeutic agent, the fluid composition within the reservoir 12 may contain a second, third, fourth, etc. therapeutic agent. Alternatively, the device 30 may have more than one reservoir 12 for housing additional compositions comprising a therapeutic agent. When the device 30 has more than one reservoir 12, the pump 40 may draw fluid from one or more reservoirs 12 and deliver the drawn fluid to the catheter 38. The device 30 may contain a valve operably coupled to the pump 40 for selecting from which reservoir(s) 12 to draw fluid. Further, one or more catheters 38 may be coupled to the device 30. Each catheter 38 may be adapted for delivering a therapeutic agent from one or more reservoirs 12 of the pump 40. A catheter 38 may have more than one lumen. Each lumen may be adapted to deliver a therapeutic agent from one or more reservoirs 12 of the device 30. It will also be understood that more than one device 30 may be used if it is desirable to deliver more than one therapeutic agent. Such therapy delivery devices, catheters, and systems include those described in, for example, copending application Ser. No. 10/245,963, entitled IMPLANTABLE DRUG DELIVERY SYSTEMS AND METHODS, filed on Dec. 23, 2003, which application is hereby incorporated herein by reference.

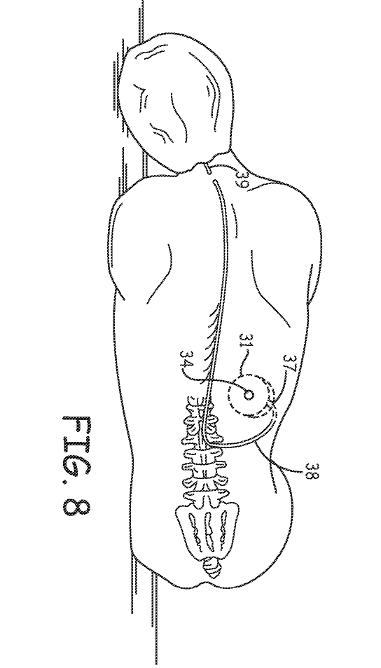

FIG. 8 illustrates a system adapted for intrathecal delivery of a composition comprising a therapeutic agent. As shown in FIG. 8, a system or device 30 may be implanted below the skin of a patient. Preferably the device 30 is implanted in a location where the implantation interferes as little as practicable with patient activity. One suitable location for implanting the device 30 is subcutaneously in the lower abdomen. According to an embodiment, catheter 38 may be positioned so that the distal end 39 of catheter 38 is located in the subarachnoid space of the spinal cord such that a delivery region (not shown) of catheter is also located within the subarachnoid space. It will be understood that the delivery region can be placed in a multitude of locations to provide direct delivery of a therapeutic agent to a multitude of locations within the cerebrospinal fluid of the patient. The location of the distal end 39 and delivery region(s) of the catheter 38 may be adjusted to improve therapeutic efficacy. While FIG. 8 shows an intraspinal target region into which a therapeutic agent is delivered, it will be understood that the therapeutic agent may be delivered anywhere within a patient's body where delivery via an infusion system may be desirable. For example, the therapeutic agent may be delivered perispinally, intraparenchymally, intracerebroventricularly, intracardially, pericardially, intrahepaticly, perineurally, etc.

Any therapeutic agent that may be formulated in a depot and placed into a target region of a patient and may be delivered to the target region by an implantable infusion device may be used in a therapy in accordance with the teachings of this disclosure. Exemplary agents that may be delivered from an implantable infusion device include agents for treating spasticity, such as baclofen; and agents for treating pain, such as opioid agonists (including morphine and sufentanil), local anesthetics (including bupivicaine), alpha2-agonists (including clonidine), gabapentin, TNFα inhibitors, norepinephrine and serotonin reuptake inhibitors, and tricyclic antidepressants (including desipramine).

Table 2 provides a representative list of agents that may delivered from an implantable infusion device to treat a disease. Exemplary daily doses of the therapeutic agent, and the route of delivery and disease to be treated are also provided in Table 2.

TABLE 2
Exemplary agents for use with implantable
infusion device therapy

| | | | | Disease to |
|---|---|---|---|---|
| Therapeutic agent | Daily dose | | Route of delivery | be treated |
| baclofen | 0.1 to 1.5 | mg/day | Intrathecal | Spasticity |
| morphine | 1 to 15 | mg/day | Intrathecal | Pain |
| sufentanil | 1 to 50 | mg/day | Intrathecal | Pain |
| bupivacaine | 2 to 30 | mg/day | Intrathecal | Pain |
| clonidine | 0.1 to 1.0 | mg/day | Intrathecal | Pain |
| gabapentin | 1 to 60 | mg/day | Intrathecal | Pain |
| desipramine | 1 to 60 | mg/day | Intrathecal | Pain |

It will be understood that if a pharmaceutical depot configured to release a therapeutic agent at a particular release rate successfully improves a symptom of the disease in a patient, an infusion device 30 configured to deliver therapeutic agent at a substantially similar rate may be selected or programmed accordingly. It will be further understood that the concentration of the therapeutic agent in a reservoir 12 of the infusion device 30 may be adjusted to obtain such a substantially similar release rate.

Dosage

Effective dosages for use in methods as described herein can be determined by those of skill in the art, particularly when effective systemic dosages are known for a particular therapeutic agent. Dosages may typically be decreased by at least 90% of the usual systemic dose if the therapeutic agent is provided in a targeted fashion. In other embodiments, the dosage is at least 75%, at least 80% or at least 85% of the usual system dose for a given condition and patient population. Dosage is usually calculated to deliver a minimum amount of one or more therapeutic agent per day, although daily administration is not required. If more than one pharmaceutical composition is administered, the interaction between the same is considered and the dosages calculated. Intrathecal dosage, for example, can comprise approximately ten percent of the standard oral dosage. Alternatively, an intrathecal dosage is in the range of about 10% to about 25% of the standard oral dosage.

What is claimed is:

1. A method for identifying whether a patient having a disease is a candidate for delivery of a therapeutic agent via an implantable infusion device to a targeted location for treatment of the disease, the method comprising:
placing in a patient a pharmaceutical depot comprising the therapeutic agent, the depot being placed in proximity to the targeted location; and
determining whether the symptom has improved following placement of the depot in the patient,
wherein the patient is identified as a candidate for delivery of a therapeutic agent via an implantable infusion device if the symptom is improved.

2. The method of claim 1, wherein the pharmaceutical depot is configured to release the therapeutic agent in the target region at a rate similar to that achievable by the implantable infusion device.

3. The method of claim 2, wherein the pharmaceutical depot releases the therapeutic agent at the release rate for at least about three days after being placed in the patient.

4. The method of claim 2, wherein the pharmaceutical depot releases the therapeutic agent at the release rate for at least about five days after being placed in the patient.

5. The method of claim 2, wherein the pharmaceutical depot releases the therapeutic agent at the release rate for at least about one week after being placed in the patient.

6. The method of claim 2, wherein the pharmaceutical depot releases the therapeutic agent at the release rate for at least about two weeks after being placed in the patient.

7. The method of claim 2, wherein the pharmaceutical depot releases the therapeutic agent at the release rate for at least about one month after being placed in the patient.

8. The method of claim 1, wherein placing the pharmaceutical depot in proximity to the targeted location of the patient comprises placing the depot perispinally.

9. The method of claim 8, wherein placing the pharmaceutical depot in proximity to the targeted location of the patient comprises placing the depot epidurally.

10. The method of claim 1, wherein placing the pharmaceutical depot in proximity to the targeted location of the patient comprises placing the depot intrathecally.

11. A method comprising:
placing in a patient a pharmaceutical depot comprising a therapeutic agent, the depot being placed in proximity to a target location;
determining whether a symptom of the patient is improved following placement of the depot in the patient; and
implanting an infusion device in the patient so that the therapeutic agent is delivered from the infusion device to the target location if the symptom is improved.

12. The method of claim 11, wherein the pharmaceutical depot is configured to release the therapeutic agent in the target region at a rate similar to that achievable by the implantable infusion device.

13. The method of claim 12, wherein the pharmaceutical depot releases the therapeutic agent at the release rate for at least about one week after being placed in the patient.

14. The method of claim 11, wherein placing the depot in proximity to the target region of the patient comprises placing the depot perispinally.

15. The method of claim 14, wherein implanting the infusion device in the patient so that the therapeutic agent is delivered to the target location comprises delivering the therapeutic agent intrathecally.

16. The method of claim 14, wherein the disease is spasticity.

17. The method of claim 14, wherein the disease is pain.

18. A method for identifying whether a patient having a disease is a candidate for implantable infusion device therapy, the method comprising:
placing in a patient a first pharmaceutical depot comprising a first therapeutic agent, the depot being placed in proximity to a targeted location; and
determining whether a symptom of the disease has improved following placement of the first depot in the patient;
if the symptom has not improved,
(i) placing in proximity to the targeted location a second pharmaceutical depot comprising a second therapeutic agent, and
(ii) determining whether the symptom of the disease has improved following placement of the second depot in the patient,
wherein the patient is identified as a candidate for delivery of the first or second therapeutic agent via an implantable infusion device if the symptom has improved.

19. A method for identifying whether a patient having a disease is a candidate for implantable infusion device therapy, the method comprising:
placing in the patient a first pharmaceutical depot configured to release a therapeutic agent at a first rate, the depot being placed in proximity to a targeted location; and
determining whether a symptom of the disease has improved following placement of the first depot in the patient;
if the symptom has not improved,
(i) placing in proximity to the targeted location a second pharmaceutical depot configured to release the therapeutic agent at a second rate, and
(ii) determining whether the symptom of the disease has improved following placement of the second depot in the patient,
wherein the patient is identified as a candidate for delivery of the therapeutic agent via an implantable infusion device if the symptom has improved.

20. A method comprising:
placing in a patient a first pharmaceutical depot configured to release a therapeutic agent at a first rate, the depot being placed in proximity to a targeted location; and
determining whether a symptom of a disease of the patient has improved following placement of the first depot in the patient;
if the symptom has not improved,
(i) placing in proximity to the targeted location a second pharmaceutical depot configured to release the therapeutic agent at a second rate, and
(ii) determining whether the symptom of the disease has improved following placement of the second depot in the patient; and
if the symptom has improved, implanting an infusion device in the patient so that the therapeutic agent is delivered from the infusion device to the target location.

21. The method of claim 20, wherein the infusion device is configured to release the therapeutic agent at a rate substantially similar to the first rate if the symptom improved after placement of the first depot in the patient.

22. The method of claim 20, wherein the infusion device is configured to release the therapeutic agent at a rate substantially similar to the second rate if the symptom improved after placement of the second depot in the patient.
